(19) United States
(12) Patent Application Publication
(10) Pub. No.: US 2009/0216144 A1
(43) Pub. Date: Aug. 27, 2009

(54) Hopping methods for the detection of QRS onset and offset

(76) Inventor: Bruce Hopenfeld, Salt Lake City, UT (US)

Correspondence Address: ROSENBERG, KLEIN & LEE, 3458 ELLICOTT CENTER DRIVE-SUITE 101, ELLICOTT CITY, MD, 21043, US

(21) Appl. No.: 12/071,618

(22) Filed: Feb. 25, 2008

(51) Int. Cl. A61B 5/0456

(52) U.S. Cl. 600/521

(57) ABSTRACT

Disclosed are systems and methods for detecting the QRS portion of an electrocardiogram and also for detect QRS onset/offset points. QRS complexes are identified by testing selected waveform samples to determine if they are in a high curvature region. If a region passes the high curvature test, a verification test is applied to adjacent samples to confirm that the region is within a QRS complex. The search for a QRS onset point begins at an adaptive distance from this region. The curvature of selected regions ahead in the search direction is examined. The search moves past all such high curvature regions. The onset point is then located by searching for a region that is “flat” in either of two senses. First, a region may be locally flat (i.e. small first derivative) in an absolute sense. Second, a region is flat in a relative that further movement away from the interior of the QRS (e.g. toward “earlier” samples) does not result in a significant change in the value of the first derivative. The search is performed by hopping through the waveform samples. Once an onset point is located, the search for an offset point proceeds in a similar manner from a starting point that is an adaptively determined distance from the onset point.

### FIELD OF USE

This invention is in the field of medical device systems that process electrocardiograms.

### BACKGROUND OF THE INVENTION

Heart disease is the leading cause of death in the United States. A heart attack, also known as an acute myocardial infarction (AMI), typically results from a blood clot or “thrombus” that obstructs blood flow in one or more coronary arteries. AMI is a common and life-threatening complication of coronary artery disease. Coronary ischemia is caused by an insufficiency of oxygen to the heart muscle. Ischemia is typically provoked by physical activity or other causes of increased heart rate when one or more of the coronary arteries is narrowed by atherosclerosis. AMI, which is typically the result of a completely blocked coronary artery, is the most extreme form of ischemia. Patients will often (but not always) become aware of chest discomfort, known as “angina”, when the heart muscle is experiencing ischemia. Those with coronary atherosclerosis are at higher risk for AMI if the plaque becomes further obstructed by thrombus.

There are a number of portable monitors that attempt to detect AMI. Monitors that include wearable sensors (e.g. a medical-vest with electrodes) may be somewhat inconvenient for patients. Chronically implanted sensors provide the possibility for continuous monitoring without many of the inconveniences associated with wearable monitors. One type of implantable monitor includes an electrode chronically implanted within the heart, as described in U.S. Pat. No. 6,609,023 to Fischell et al. An intracardiac electrode may provide a strong signal at the cost of requiring intracardiac implantation. Another type of implantable monitor relies upon subcutaneous electrodes, which are less invasive, but receive smaller amplitude signals compared to intracardiac electrodes.

Song et al. describe a small rectangular device that may be implanted in the left precordial region to detect acute ST shifts. (“The feasibility of ST-segment monitoring with a subcutaneous device”, J Electrocardiol. 2004; 37 Suppl: 174-9). The ST segment is defined as a specified rate adjusted temporal offset from the peak of the R wave. Measuring the ST segment in this fashion may not be optimal in cases where the QRS complex shape changes due to many factors (e.g. axis) shifts. Also, patients with chronic ischemia and/or other conditions may exhibit abnormal and sharp ST upstrokes and downstrokes as measured on the body surface, so that the detected ST level could become sensitive to the exact region where it is measured in the ST segment. ST segment changes due to increased heart rate (and other factors) could thus cause somewhat undesired fluctuations if the ST segment is measured at a certain offset from the R wave.

A similar scheme ST measurement scheme is disclosed in U.S. Pat. No. 6,397,100 to Stadler and Shannon. The R wave peak is detected and the ST segment is defined as a certain number of samples after the R wave peak. The isoelectric point for the QRST complex, which serves as the basis for defining ST segment potential, is found by searching backwards from the R wave peak for a minimum slope: “Beginning at 5 samples (40 ms) before the R-peak sample, the algorithm marches backwards in steps of two samples, searching for a local minimum in the absolute slope between successive samples . . . . The search concludes when a local minimum of slope is found, or when the search reaches the beginning of the stored data points of the PQRST complex. The isoelectric point 1 value is then averaged over 2 samples (16 ms).” This type of searching may be relatively computationally expensive and may also prove problematic with certain types of PQ wave shapes.

Hayn et al., (“Automated QT Interval Measurement from Multilead ECG Signals”, Comp. Card. 33:381-4, 2006) describe a method for detecting Q wave onset by first selecting a coarse QRS onset point by comparing the change in the signal over time (i.e. gradient) to a threshold. This coarse onset point was then fine tuned by a stepwise decrease of the threshold. For each such step, a “possible onset point was calculated and the mean range curve value right before and after this possible point was determined. The point with the lowest ratio in between these mean values was chosen as the exact onset point.” The “range curve” is the difference in signal amplitude, so a ratio of range curves before and after a point is a ratio of first derivative/gradient quantities. T wave offsets were also found by this method, as well as by fitting a Gaussian curve to the T wave and defining the offset with respect to the standard deviation of the curve.

Sun et al. (“Characteristic wave detection in ECG signal using morphological transform”; BMC Cardiovasc Disord. 2005; 5: 28) describe a multi-scale derivative method for locating QRS (and P and T) wave fiducial points. “Long distance” derivatives/differences (e.g. f(x+n)−f(x), where n>1) are taken both before and after each candidate onset point and the difference between these derivatives is calculated to form a type of second derivative/difference which the authors term a “multiscale morphological derivative transform.” QRS onset or other fiducial points are defined as the maxima or minima of this “transform.” Kemmelings et. al. describe a QRS onset/offset detection scheme that involves summing the absolute value of the first derivative (difference) and then taking a “long distance” derivative of this summed signal, to find where it abruptly changes (over a large scale). (“Automatic QRS onset and offset detection for body surface Q RS integral mapping of ventricular tachycardia.”; IEEE Trans Biomed Eng. 1994; 41:830-836).

Zong et al. (“A robust open-source algorithm to detect onset and duration of QRS complexes”, Computers in Cardiology, 2003, Issue, 21-24 Sep. 2003, Page(s): 737-740) describe a method for detecting QRS onset and offset points by applying thresholds to the values of a function that corresponds to the “distance” along the signal; the function has the form (D²+(Δs/Δt)²)^{0.5}, where D is a constant and Δs is the difference between successive samples of an ECG signal and Δt is the time between samples (i.e. the inverse of the sampling frequency). This function is essentially a discrete version of the calculus formula for distance along a curve, which is based on the first derivative of the curve.

Christov describes a method for QRS detection that involves the combination of adaptive slope criteria, adaptive “integrating threshold for high-frequency components”, and adaptive criteria pertaining to the duration between beats. (Christov, I., “Real time electrocardiogram QRS detection using combined adaptive threshold.” BioMedical Engineering OnLine 2004, 3:28.) Christov does not describe any methods to detect QRS onset or offset points.

U.S. Pat. No. 5,758,654 describes a method for QRS onset/offset detection. First, a QRS peak is located based upon, among other things, second derivative criteria. Adjacent QRS extrema (i.e. peaks) are located, apparently by stepping through consecutive waveform samples, and selected as coarse QRS onset/offset times. The coarse onset/offset times are subsequently refined based on criteria related to the region of the waveform bounded by optimal QRS onset/offset points.

U.S. Pat. No. 6,650,931 to McClure et al. describes search techniques for finding the onsets and offsets of various cardiac events. An amplitude based test is applied to detect onset points. For offset points, both amplitude and first derivative based tests are applied to determine offset points. After an onset point is found, a search pointer is advanced a certain distance and then a sequential search is performed for offset points; the search pointer is advanced from the onset point by the shortest expected duration of the event being analyzed.

U.S. Pat. No. 6,625,490 931 to McClure et al. describes a “System and method of automatically adjusting sensing parameters based on temporal measurement of cardiac events.”

It is known in the art to square the derivative of an electrocardiogram signal for the purposes of making the signal positive and emphasizing the high frequency aspects of the signal. Friesen et al. describe an algorithm that involves computing, scaling and smoothing first and second derivatives and adding the two together. (Friesen, G. M., Jannett, T. C., Jadallah, M. A., Yates, S. L., Quint, S. R., Nagle, H. T. 1990. “A comparison of the noise sensitivity of nine QRS detection algorithms.” IEEE Trans. Biomed. Eng., BME-37: 85-97). The resulting waveform is scanned until a sample exceeds a threshold, in which case subsequent points are also checked against the threshold; if a certain percentage of subsequent samples exceeds the threshold, a QRS complex has been detected. Taken together, all of the above mentioned steps are somewhat expensive from the computation and memory standpoints.

Arzeno et al. describe a number of QRS detection algorithms in a recent paper. (Analysis of First-Derivative Based QRS Detection Algorithms, IEEE Transactions on Biomedical Engineering, Vol. 55, No. 2, 2008.) Arzeno et al. describe one technique that involves finding a peak in a transformed signal, and starting the search for a real peak in an QRS at a location where the transformed signal is one half its peak value.

Despite all of the foregoing work that has been done, there is still a need for an effective, computationally efficient method for detecting the QRS complex and QRS onset and offset points.

### SUMMARY OF THE INVENTION

An embodiment of the present invention comprises a lead and a processor that computes waveforms from electrocardiograms sensed by the lead. A waveform is scaled so that its overall amplitude (maximum-minimum voltage) is set to a known value. QRS complexes are identified by testing selected waveform samples to determine if they are in a high curvature region. If a region passes the high curvature test, indicating it may be within a QRS, a verification test is applied to adjacent samples to confirm that the region is within a QRS complex.

From this region, which will be referred to as the QRS search point, the algorithm moves toward the QRS onset point by first moving an adaptively determined distance toward the QRS onset point. The adaptively determined distance is based on the expected distance between QRS search points and onset points as determined in past searches. After moving this distance, the routine attempts to find regions of high curvature/high frequency ahead by examining the curvature at various regions that are separated from one another. (“Ahead” is taken in the sense of the search direction.) If it finds such regions, it hops past them towards the QRS onset point.

When the routine has hopped past all such high curvature/high frequency regions, the region of the coarse onset point is located by executing further hops until a region is located that is “flat” in either of two senses. First, the region may be locally flat (i.e. small first derivative) in an absolute sense. However, for certain QRS morphologies, there may not be a region that is locally flat in an absolute sense, so the “deceleration”/second derivative of the waveform is examined: if the slope of the waveform begins to change very slowly, the region is flat in a relative sense and taken as the QRS onset point. More particularly, the region of signal deceleration is flat in the sense that further movement away from the interior of the QRS (e.g. toward “earlier” samples) does not result in a significant change in the value of the first derivative.

The search for a flat region proceeds by hopping through the waveform, landing on regions that are separated from one another. At any such region, various regions ahead are examined to determine if there is a significant change in the first derivative somewhere ahead of the current region. Upon detecting such a significant change at a particular region ahead, the routine hops to that region and continues the search by applying the locally flat test mentioned above. If the locally flat test fails, then the deceleration test is applied once again. To reiterate, the search stops (and a coarse onset point is thus located) when a current region passes either the locally flat test or the deceleration test.

After an onset point is located, a search for the offset point begins at a region that is a adaptive distance from the onset point. The distance is adaptively adjusted based on the QRS duration of past QRS complexes. The offset point search is otherwise similar to the onset point search. After locating the coarse onset point, the final onset point is determined by fine tuning according to first derivative criteria.

### BRIEF DESCRIPTION OF THE DRAWINGS

FIG. 1 illustrates a system for the detection of a cardiac event and for warning the patient that a medically relevant cardiac event is occurring;

FIG. 2 is a block diagram of an implanted cardiac diagnostic system in FIG. 1.

FIGS. 3-6 are flowcharts of the preferred QRS detection method and the QRS onset/offset detection method implemented by the diagnostic system shown in FIG. 2. FIG. 3 is a high level flowchart of both QRS detection methods and onset/offset detection methods integrated into a single system. FIG. 4 is a flowchart of the QRS complex routine, which results in the selection of a single sample within each QRS. FIG. 5 is a flow chart that shows the high level steps for computing QRS onset and offset times. FIGS. 6a and 6b together are a block diagram of the details of the QRS onset/offset detection routine, which is fed a starting search point from the high level routine shown in FIG. 5.

FIG. 7 shows graphs of the initial portion of a QRS complex, its corresponding first derivative, and its corresponding curvature function value, respectively.

FIG. 8 shows a QRS complex of an ectopic beat.

FIGS. 9a-9l show computer code of the preferred embodiment of the present invention.

### DETAILED DESCRIPTION OF THE INVENTION

“Lead” means at least two sensors that are configured to detect the electrical potential between two points.

A “function” is used in a mathematical sense as a mapping of one or more variables to a value that is computable without performing a search.

A “function value at a region” is a mapping of a waveform value of a region and possibly other regions to a value. For example, the local first derivative of a region A is a function value at region A, and the ratio of the first derivative at region A to the first derivative at region B is also a function value at region A (or B).

“Curvature function” means a mapping of a region of a waveform to a value (or values) that is correlated with the curvature of the region. Examples of curvature functions include local or “long distance” second derivatives and such second derivatives raised to a power.

The “derivative” at a point of a waveform consisting of points sampled at a constant sampling rate is equivalent to the difference in the value of the waveform around that point. For example, a derivative at sample number x is D=W(x+n)−W(x−n), where W(x) is the value of a time series waveform at sample/point x, and where n is an integer that corresponds to the scale of the derivative. A “long distance” derivative is broadly meant to include cases where n is greater than 1.

A “region” of a discrete waveform is the set of one or more time points within a time interval, whether or not those time points coincide with samples. For example, if a waveform consists of samples at times 1 and 2, the region [1 2] includes a point at time 1.5. A region can be a single sample.

A “point” is identical to a sample, i.e. a discrete value of a waveform.

An “ecg” broadly refers to any type of recording of a person's cardiac electrical activity. Without limiting the foregoing, an “ecg” may be recorded from any combination of electrodes inside a heart, inside a body or outside of a body.

A “test” is one or more steps involved in the application of a decision rule to at least one numeric value. A test may be applied to a function value or a portion of a waveform. In the latter case, the test is the compound procedure of computing a function (e.g. taking a first derivative) and a applying a decision rule to the function output. For example, the entire test may be stated as: “if the first derivative is less than 10, the region is considered flat.” A test may consist of the application of a number of (lower level) tests.

A “stopping test” is positive at a region when a search criterion (or criteria) is satisfied at the region. A search ends upon a positive stopping test.

A “QRS marker point” is any point within or adjacent to a QRS that is defined according to some criteria than enables the point to be searched for. Examples of QRS marker points include QRS onset, QRS offset, and R wave peak. QRS marker points may or may not be QRS fiducial points.

A “QRS fiducial point” is a point that is specified by any combination of maximum or minimum tests applied directly to any combination of amplitude, first derivative and second derivative values.

A region is “moved to” or “hopped to” from a current point during a search for a marker point if it is determined that the region is closer to the marker point than the current point is to the marker point. As an example, if the current point is 20, and the curvature function is examined at point 30 and is determined to be large, so that it is assumed that the marker point is at least 5 samples beyond 30, a hop is made to 35. Sample 30 has been (implicitly) moved to.

“Distance” refers to a time interval.

Architecture

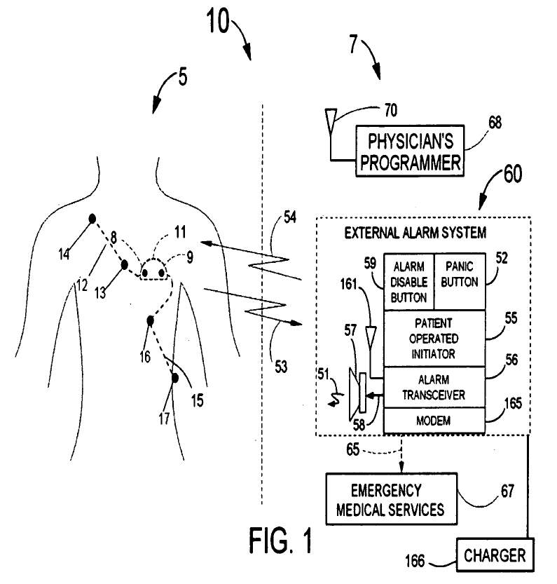

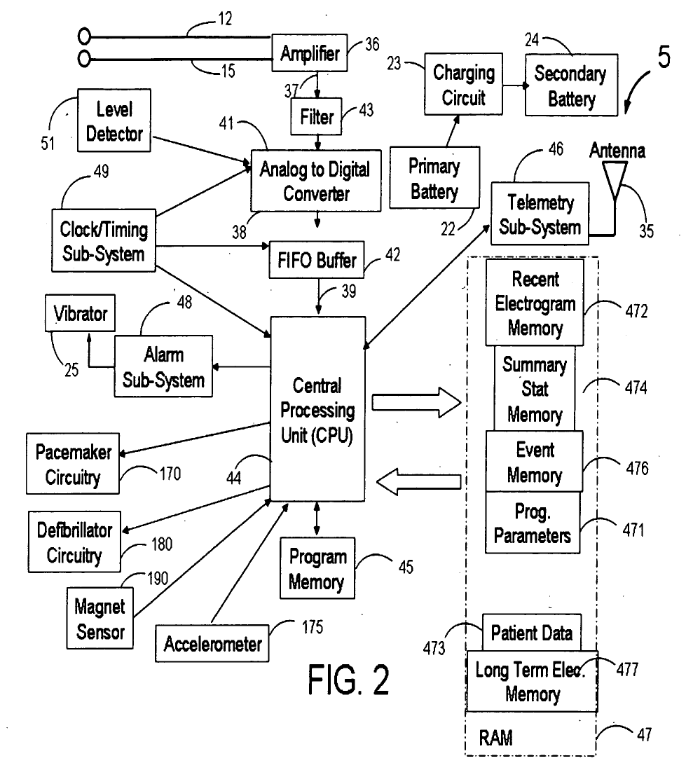

FIG. 1 illustrates one embodiment of a system 10 comprising an implanted cardiac diagnostic device 5 and external equipment 7. The battery powered cardiac diagnostic device 5 contains electronic circuitry that can detect a cardiac event such as an acute myocardial infarction or arrhythmia and warn the patient when the event, or a clinically relevant precursor, occurs. The cardiac diagnostic device 5 can store the patient's ecg for later readout and can send wireless signals 53 to and receive wireless signals 54 from the external equipment 7. The functioning of the cardiac diagnostic device 5 will be explained in greater detail with the assistance of FIG. 2.

The cardiac diagnostic device 5 receives electrical signals from subcutaneous or body surface leads 12 and 15. Right side lead 12 comprises electrodes 13 and 14 with polarity hereafter defined as the difference potential measured between electrode 13 and electrode 14. Left side lead 15 comprises electrodes 16 and 17 with polarity hereafter defined as the potential at electrode 16 minus the potential at electrode 17. The right side lead 12 measures the electrical signal between the upper right clavicle and a medial region over the region of the sternum and is therefore generally less than 15 cm long; the right side lead is approximately aligned with the long axis of the heart. The left side lead 15 measures the electrical signal between the left precordial chest region and an inferior left lateral or posterior torso position. The cardiac diagnostic device 5 is housed in a metal case 11 that can serve as another electrode. In particular, the lead 15 could effectively be temporally multiplexed so that it alternately measures the potential across electrodes 16 and 17 and the potential across the metal case 11 and the electrode 16 (or 17).

FIG. 1 also shows the external equipment 7 that consists of a physician's programmer 68 having an antenna 70, an external alarm system 60 including a charger 166. The external equipment 7 provides means to interact with the cardiac diagnostic device 5. These interactions include programming the cardiac diagnostic device 5, retrieving data collected by the cardiac diagnostic device S and handling alarms generated by the cardiac diagnostic device 5. The operation of these components is further described in U.S. patent application publication number 2004/0215092.

FIG. 2 is a block diagram of the cardiac diagnostic device 5 with primary battery 22 and a secondary battery 24. The secondary battery 24 is typically a rechargeable battery of smaller capacity but higher current or voltage output than the primary battery 22 and is used for short term high output components of the cardiac diagnostic device 5 like the RF chipset in the telemetry sub-system 46 or the vibrator 25 attached to the alarm sub-system 48. According to a dual battery configuration, the primary battery 22 will charge the secondary battery 24 through the charging circuit 23. The primary battery 22 is typically a larger capacity battery than the secondary battery 24. The primary battery also typically has a lower self discharge rate as a percentage of its capacity than the secondary battery 24. It is also envisioned that the secondary battery could be charged from an external induction coil by the patient or by the doctor during a periodic check-up.

The pairs of wires corresponding to leads 12 and 15 respectively connect to the amplifier 36, which is a multi-channel or differential amplifier. The amplified ecg signals 37 from the amplifier 36 are then filtered by a filter 43 (with a bandpass of xx-yy Hz) and then converted to digital signals 38 by the analog-to-digital converter 41, which preferably samples at a rate of 200 Hz. (For convenience, the methods of the present invention will be described with respect to a sampling rate of 250 Hz, which is the sampling rate associated with the publicly available European ST database, which may be downloaded at www.physionet.org.) The digital ecg signals 38, preferably in the form of 10 second segments acquired periodically, are buffered in the First-In-First-Out (FIFO) memory 42. Processor means shown in FIG. 2 as the central processing unit (CPU) 44 coupled to memory means shown in FIG. 2 as the Random Access Memory (RAM) 47 can process the digital ecg data 38 stored the FIFO 42 according to the programming instructions stored in the program memory 45. This programming (i.e. software), which implements the QRS onset/offset detection methods discussed below, enables the cardiac diagnostic device 5 to detect the occurrence of a cardiac event such as an acute myocardial infarction.

A level detector 51 is coupled to the analog to digital converter 41. The level detector 51 detects whether a patient's torso is upright or supine and also, if the torso is supine, the extent of its rotation with respect to the earth (e.g. patient is lying flat on his/her back, lying on his/her right side or left side.) Many MEMS based level detects which can also operationally serve as inclinometers, accelerometers, and general detectors for motion/force exist.

Additional sensors may communicate with the device 5 wirelessly through the telemetry sub-system. The data from these leads may correspond to digitized ecg signals (that have been processed by a remote subcutaneous device).

The operation of most of the components in FIG. 2 is further described in U.S. patent application publication number 2004/0215092.

In a preferred embodiment of the present invention the RAM 47 includes specific memory regions for 4 sets of ecg segment storage. These are the recent ecg storage 472 that would store the last 2 to 10 minutes of recently recorded ecg segments so that the ecg data occurring just before the onset of a cardiac event can be reviewed at a later time by the patient's physician using the physician's programmer 68 of FIG. 1. For example, the recent ecg storage 472 might contain eight 10-second long ecg segments that were captured every 30 seconds over the last 4 minutes.

A summary statistics memory 474 would provide storage for summary information, such as running averages, of various cardiac waveform feature values. A long term ecg memory 477 would provide storage for ecgs collected over a relatively long period of time. In the preferred embodiment, every ninth ecg segment that is acquired is stored in a circular buffer, so that the oldest ecg segments are overwritten by the newest one.

The telemetry sub-system 46 with antenna 35 provides the cardiac diagnostic device 5 the means for two-way wireless communication to and from the external equipment 7 of FIG. 1. Existing radiofrequency transceiver chip sets such as the Ash transceiver hybrids produced by RF Microdevices, Inc. can readily provide such two-way wireless communication over a range of up to 10 meters from the patient. It is also envisioned that short range telemetry such as that typically used in pacemakers and defibrillators could also be applied to the cardiac diagnostic device 5. It is also envisioned that standard wireless protocols such as Bluetooth and 802.11a or 802.11b might be used to allow communication with a wider group of peripheral devices.

Flowcharts

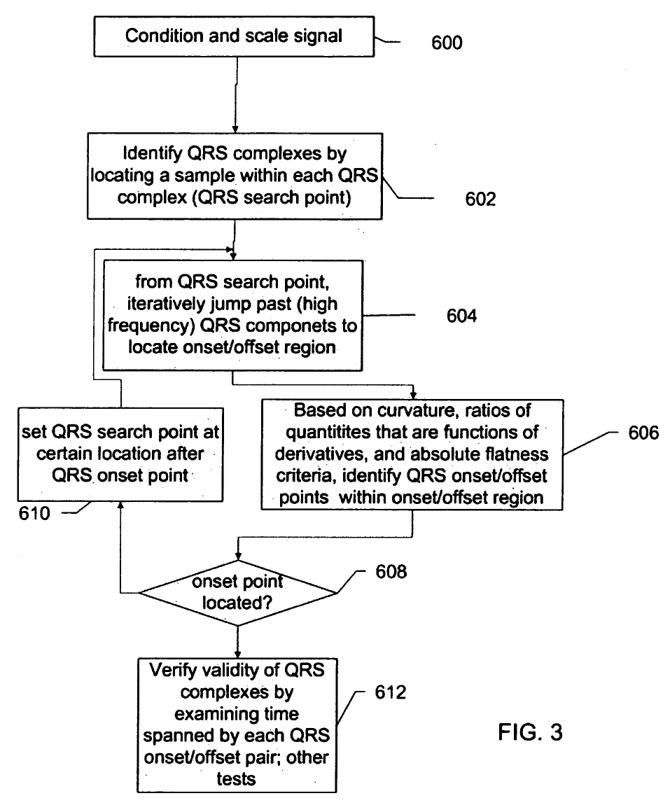

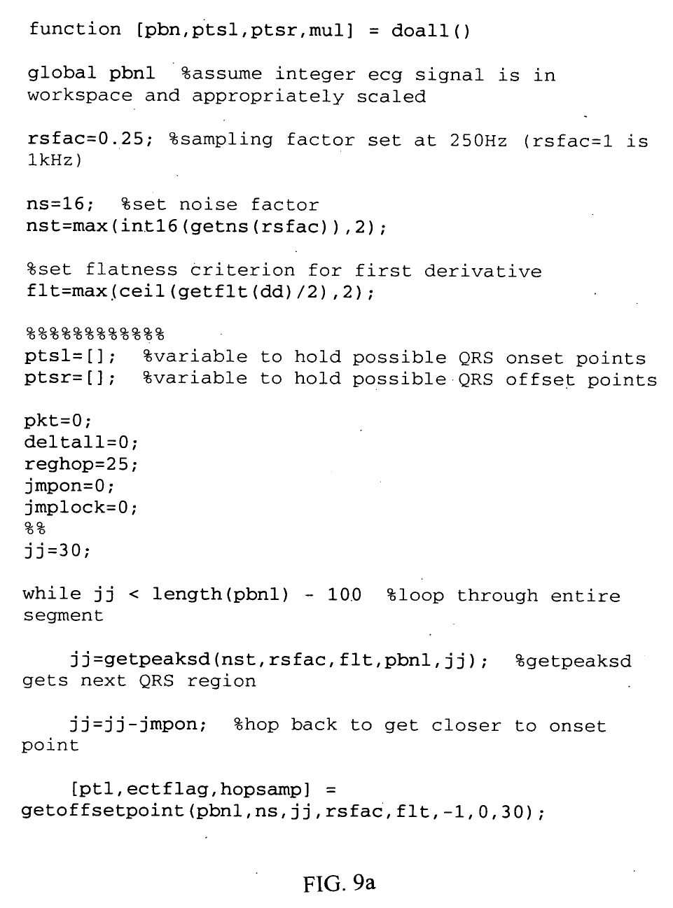

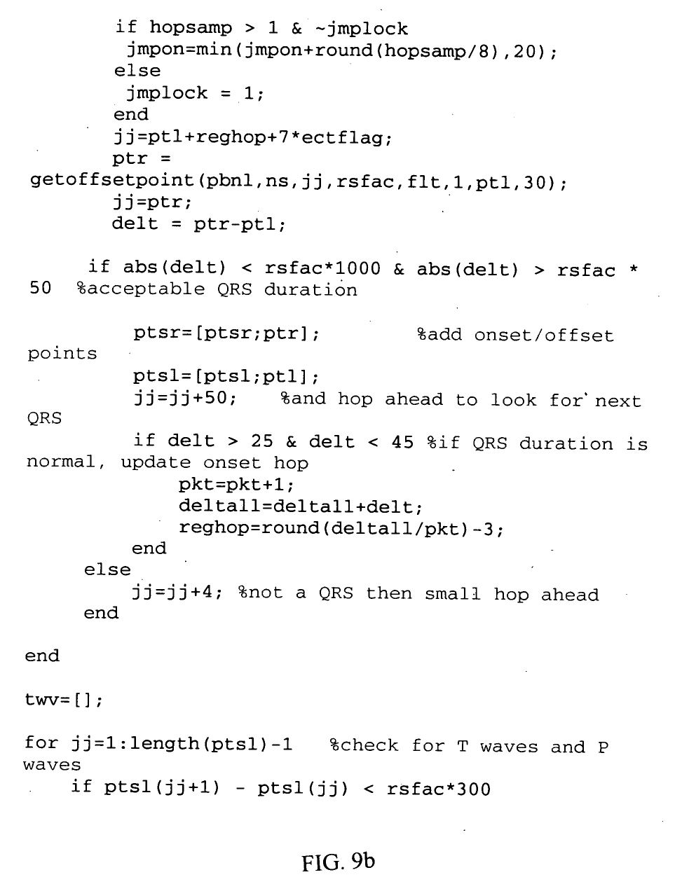

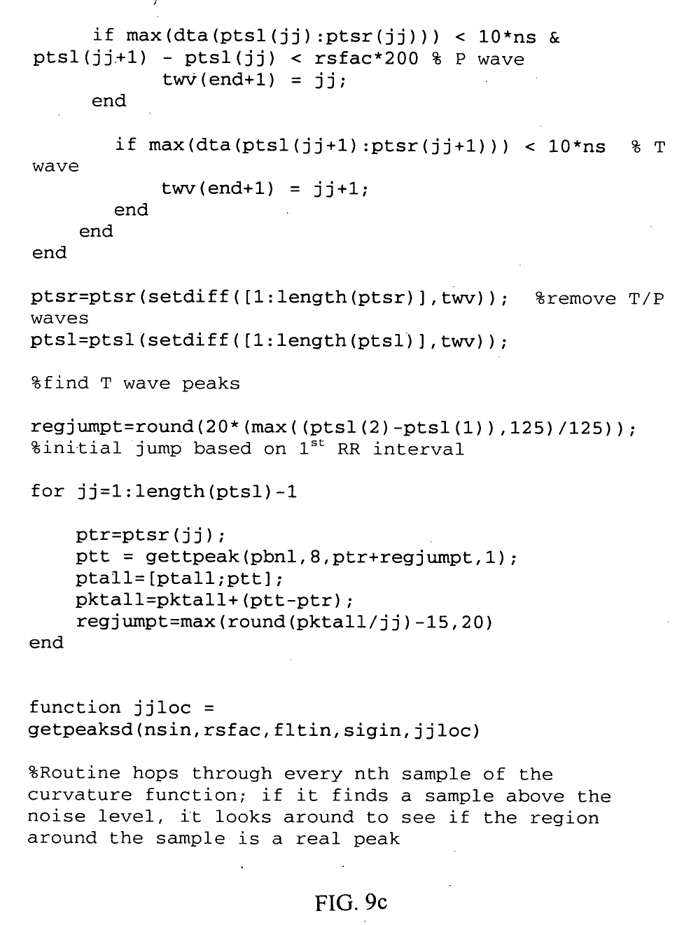

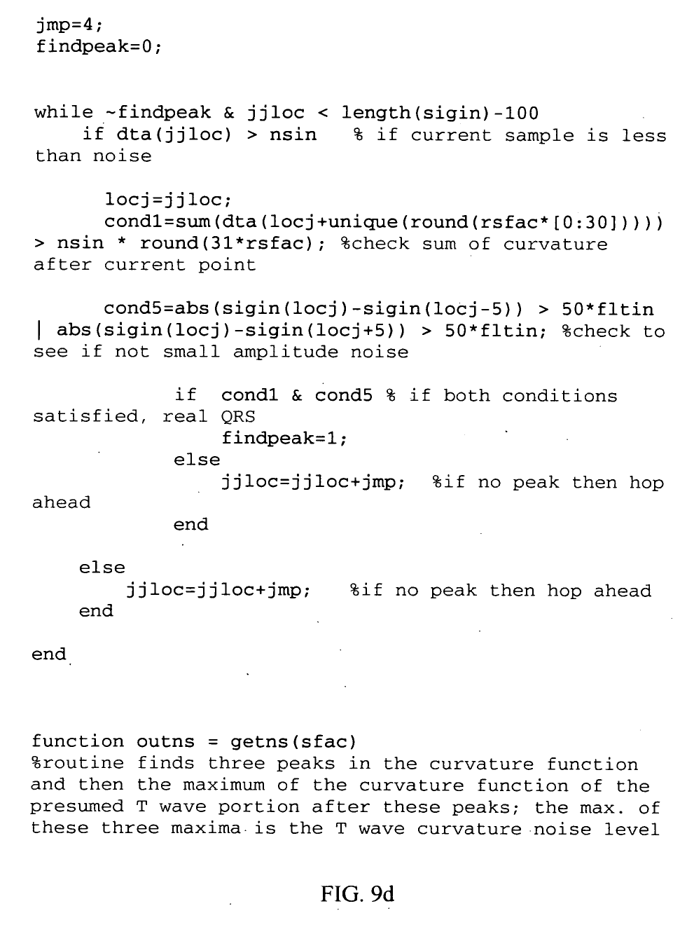

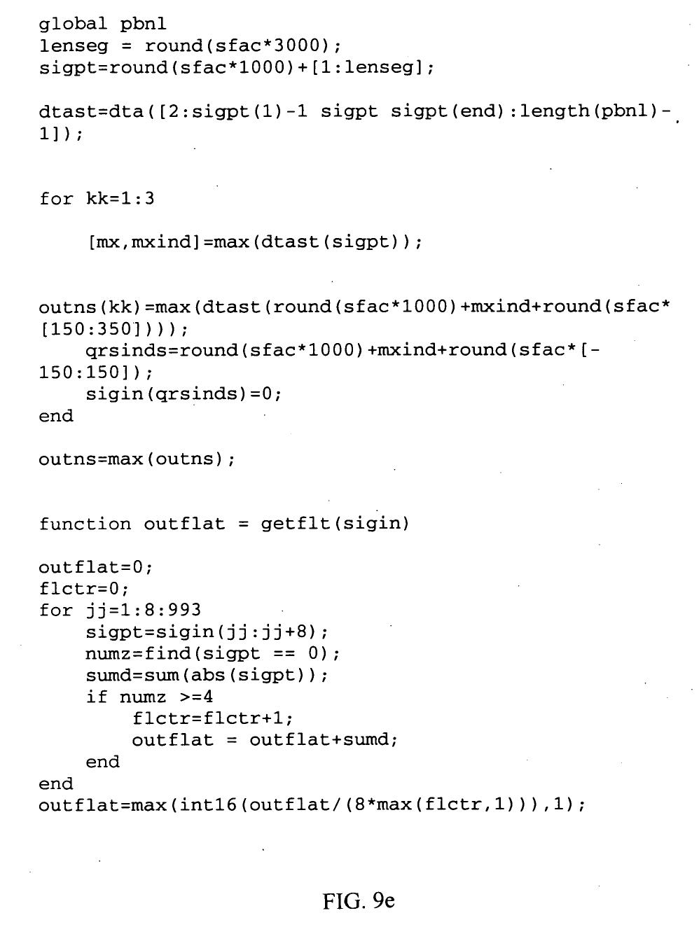

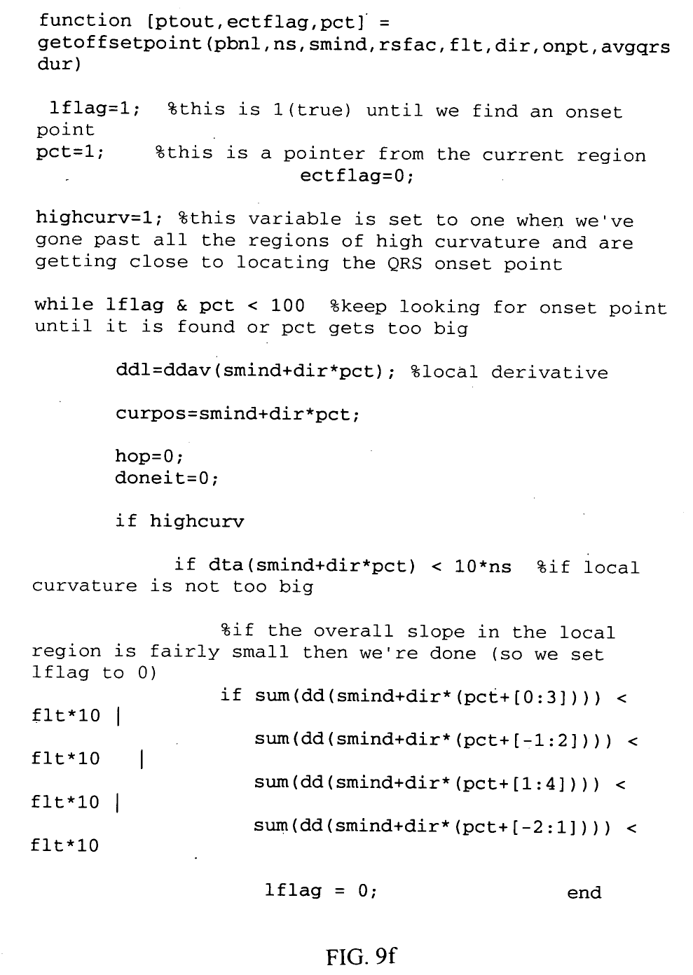

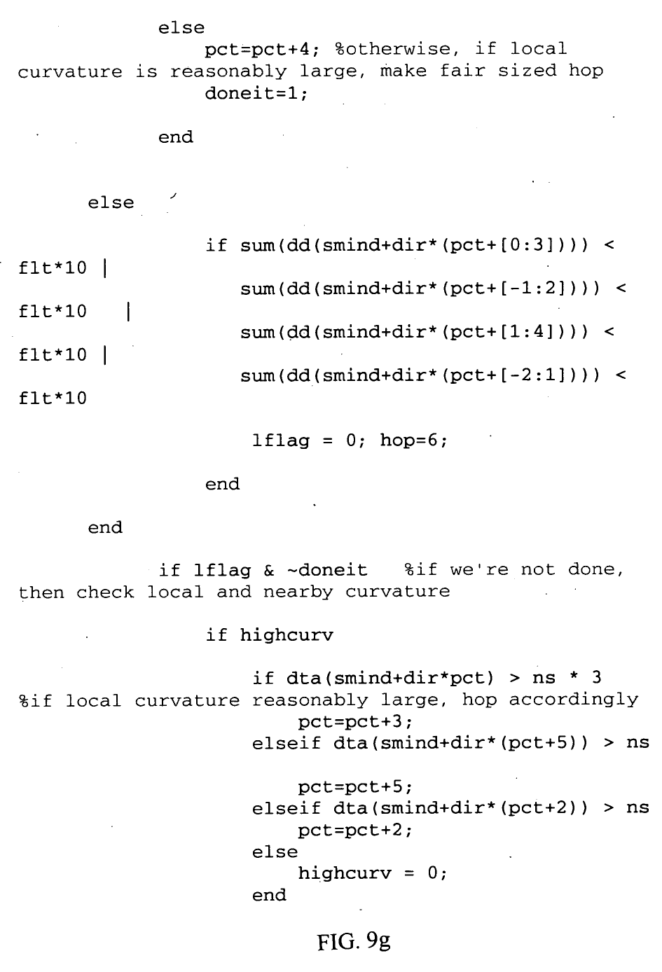

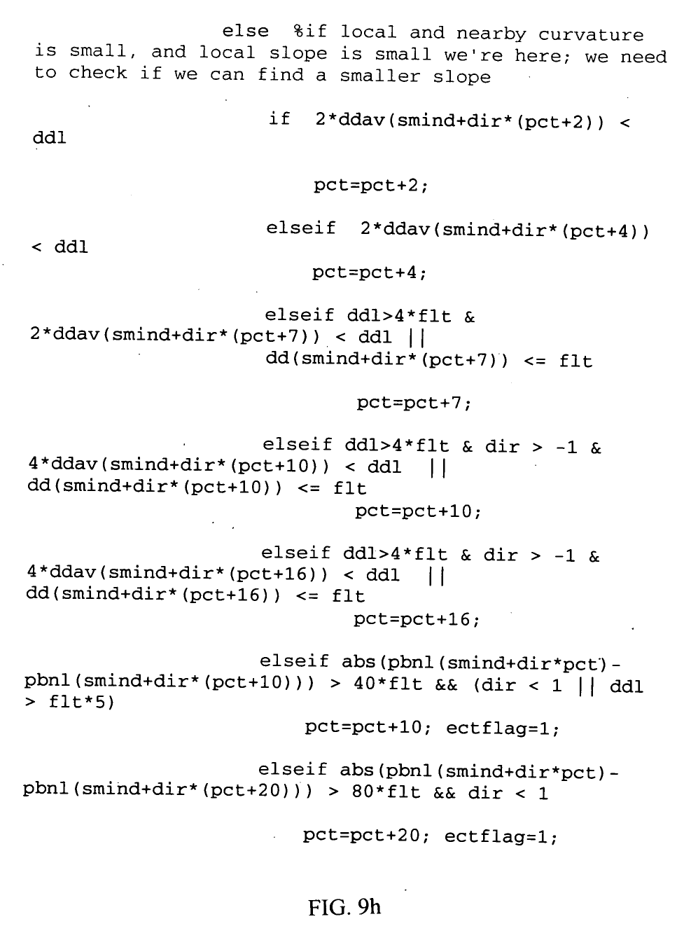

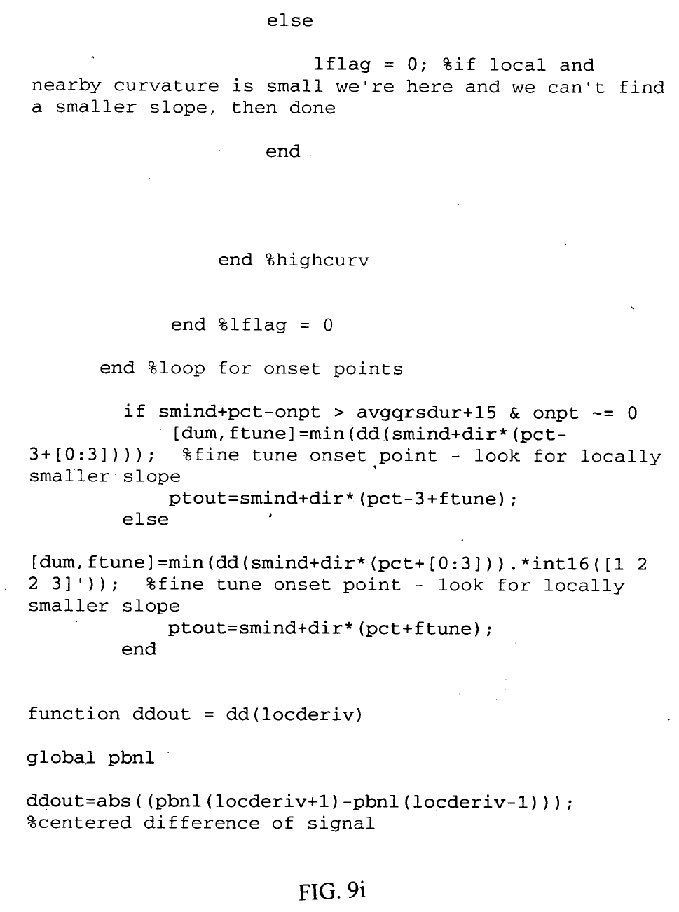

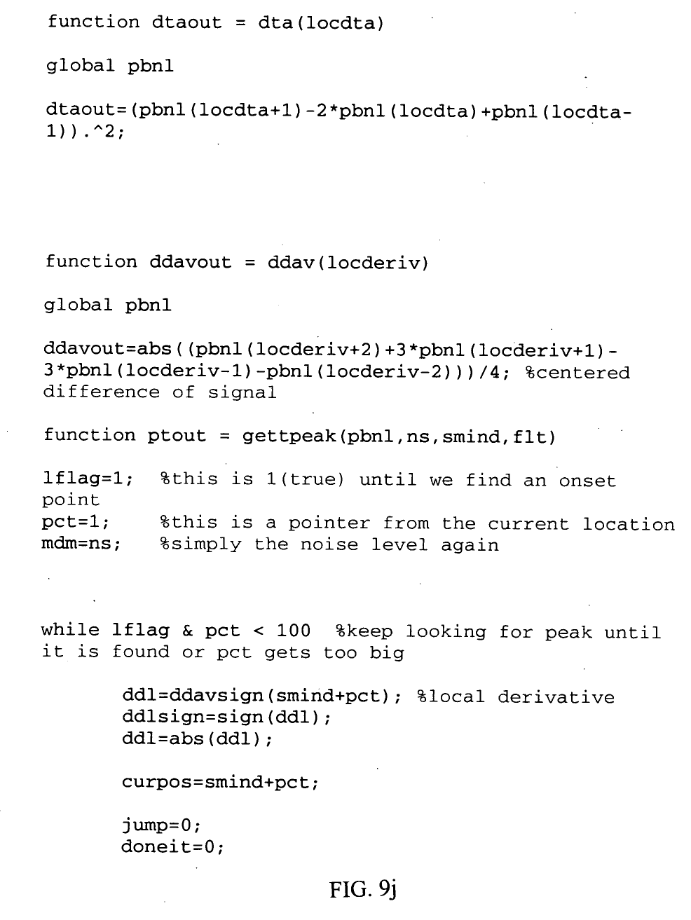

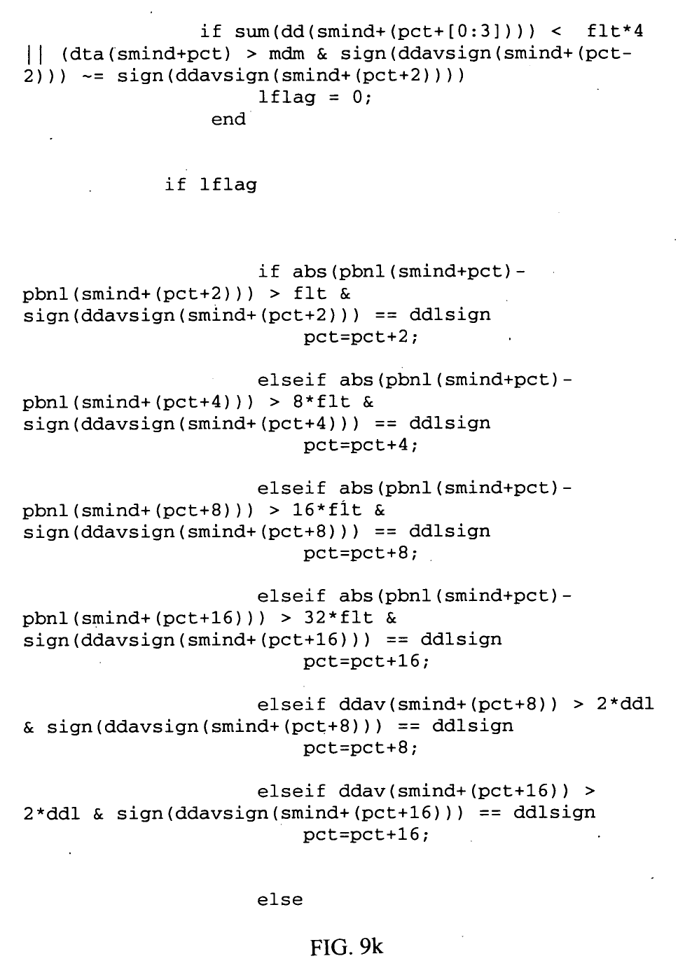

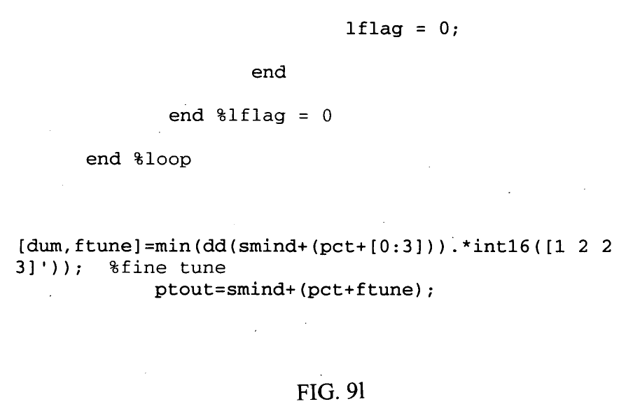

FIGS. 3-6 are flowcharts of the preferred QRS onset/offset detection method implemented by the programming instructions stored in the program memory 45 (FIG. 2). FIG. 3 is a high level flowchart of the method. The remaining flowcharts implement specific portions of the flowchart in FIG. 3. An embodiment of the methods described in FIGS. 3-6 is included in FIGS. 9a-9l in the form of Matlab (The MathWorks, Inc., Natick, Mass.) code. The code is set to operate on waveforms sampled at 250 Hz; it will be appreciated that various temporal quantities may be scaled so that the code works on waveforms sampled at different rates. (Matlab vector notation is generally used in the description below.) All arithmetic performed by the (CPU) 44 (FIG. 2) is preferably integer based, as is common practice for microcontroller based processors.

In block 600, a segment of digital ecg data 38 stored the FIFO 42 (FIG. 2) is conditioned and scaled. Scaling the ecg segment is performed because the amplitude of segments may vary for a variety of physiological or device dependent reasons. Scaling the signal so that its overall amplitude (maximum-minimum voltage) is set to a known value (e.g. 1000) allows various preset thresholds to be used across different segments. Further, and more important, scaling the signal enables certain functions of the ecg to have an appropriate dynamic range.

For example, as will be described further below, a curvature function is calculated based on the second derivative of an ecg. If the dynamic range of the curvature function is too small, e.g. it varies from 0 to 10, then information is lost compared to the case where it varies from 0 to 1000; the 0 to 1000 range provides greater discrimination between “low”, “medium” and “high” curvature parts of the curvature function.

Block 600 may also condition the signal to reduce relatively high frequency, low amplitude components (that may pass through the hardware filter 43 (FIG. 2)) by applying a simple moving average filter. This smoothing may be performed adaptively, i.e. only when the high frequency, low amplitude noise is high enough to necessitate it. Although shown as a stand-alone block, preferably, the moving average and scaling are implemented as part of the first/second derivative calculations that are performed on certain samples, as will be further described below. This eliminates the calculation of a moving average/scaling on portions of the signal that are not analyzed by the QRS onset/offset detection routine.

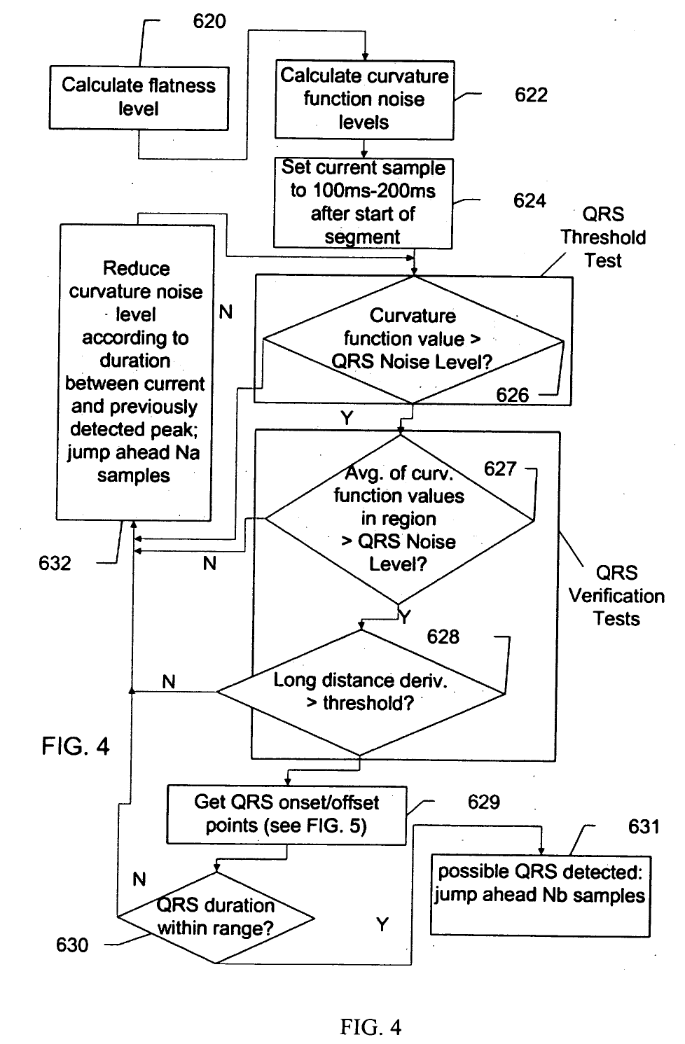

In block 602, QRS complexes are identified, preferably according to the method described in FIG. 4. According to this method, a sample within each QRS complex is located (“QRS search point”), although the region of this sample relative to any QRS fiducial point (e.g. peak R wave) is not known. In an alternative embodiment, the sample associated with a QRS fiducial is located and chosen as the QRS search point.

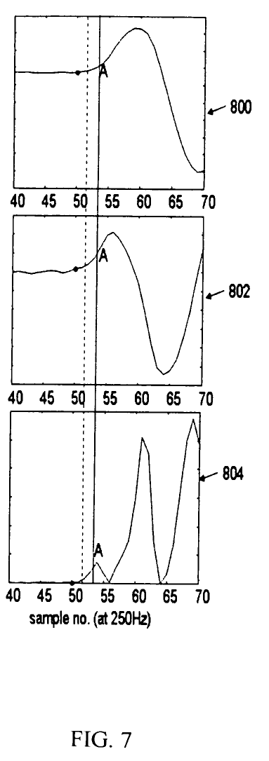

In block 604, the algorithm moves toward the QRS onset point by attempting to find regions of high curvature/high frequency in between the QRS search point and the QRS onset point and then hopping past such regions towards the QRS onset point. In other words, block 604 locates the region after a QRS onset but before any QRS maxima or minima (e.g. Q wave peak). Reference to FIG. 7 may help explain this step. FIG. 7 shows graphs 800, 802 and 804 of the initial portion of a QRS complex, its corresponding first derivative, and its corresponding curvature function value, respectively. The solid line indicates the current point, labeled A, located by the QRS detection routine. The onset/offset detection routine examines the curvature function value at point A and iteratively moves ahead (towards the onset point, in the direction of the arrow near S in graph 804) until the curvature is smaller than a threshold value (the “QRS onset/offset threshold”), which is indicated by a dotted line. (In the following description, “ahead” will always refer to the search direction, which is backward in time for onset points and forwards in time for offset points.)

Although the preferred embodiment utilizes a curvature function to find the general region of the onset point, in an alternative embodiment, a first derivative or other proxy for high frequency QRS content may be examined.

In block 606, starting from the region located in block 604, the coarse region of the onset point is located by finding a point that is “flat” in either of two senses. First, the region may be locally flat (i.e. small first derivative) in an absolute sense. However, for certain QRS morphologies, there may not be a region that is locally flat in an absolute sense, so the “deceleration”/second derivative of the signal is examined: if the slope of the signal begins to change very slowly, the region is flat in a relative sense and taken as the QRS onset point. More particularly, the region of signal deceleration is flat in the sense that further movement away from the interior of the QRS (e.g. toward “earlier” samples) does not result in a significant change in the value of the first derivative.

If an onset point was located in block 606, block 608 passes control to block 610, which sets the region of the QRS search point in the search for the QRS offset point. The region of the QRS search point is preferably adaptive based on an expected value of QRS duration given prior QRS durations. The search for offset points in blocks 604 and 606 is performed in the manner previously described with respect to the search for onset points.

Once a set of (possible) QRS onset/offset pairs has been located, in block 612, the time period between each pair to determine whether it is too long or too short to be a valid QRS. Other tests may also be performed in an adaptive manner. For example, if a test QRS onset point occurs relatively close to the prior onset (or offset) point, i.e. occurs closer than the current heart rate would suggest is a reasonable time for a beat to occur, the maximum of the curvature function between the test QRS onset point and its associated offset point may be found and compared to a threshold. If the maximum is too low, the test onset point may represent a T wave or other noise and is discarded. If the test onset point in reality represents the onset of an ectopic beat, the maximum of the curvature function will generally exceed a well chosen threshold and the test onset point will be accepted as a valid QRS.

FIG. 4 is a flowchart of the QRS complex routine, which results in the selection of a single sample within each QRS. The region of the sample within the QRS (e.g. relative to a fiducial point such as peak R wave) is not determined in the preferred embodiment.

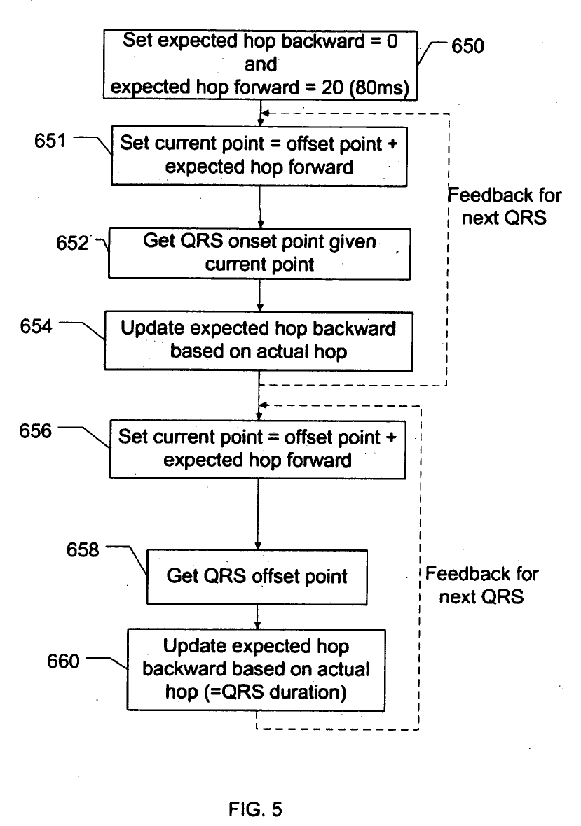

Blocks 620 and 622 are preliminary steps that result in the computation of parameter thresholds that are relevant both to QRS detection and QRS onset/offset detection, as will be described with reference to the flowchart shown in FIG. 5. Depending on the circumstances, blocks 620 and 622 may not be performed for every segment but instead may be performed periodically, such as once an hour.

In block 620, a flatness level is determined. The flatness level is the level at which the first derivative of the waveform is considered to be flat. The flatness level (“Flat”) determined in block 620 is used in connection with detecting QRS onsets and offsets and possibly in other circumstances, as will be described below. In the preferred embodiment, a single flatness level applies to all of these uses. In an alternative embodiment, different flatness thresholds may be determined for different uses (e.g. a different flatness threshold may be applied to QRS onset points than QRS offset points).

In any event, Flat in block 620 is determined by examining the first derivative of a certain number of consecutive samples (corresponding to approximately 4 seconds of waveform data). Consecutive sets of 8 first derivatives are examined to determine if there are at least 4 zeros in any particular set of 8. If so, a locally flat region has been detected, and the fluctuation of the first derivative in this region (i.e. in the set of 8 samples), provides information regarding what is considered flat. Block 620 computes the average of the first derivative across all such locally flat regions, and this average is Flat.

A few alternate embodiments for determining flatness will be described. According to one alternative, a flatness level may be empirically determined. For example, if the signal dynamic range is scaled to 1000 (as was discussed with reference to block 600 in FIG. 3), a flatness level of 2 appears to be a reasonable value. In another alternate embodiment, the flatness level for QRS onset may be determined by locating the Q wave inflection point (in the region of point A in FIG. 7), and then finding the minimum running average of the first derivative of 12 ms (3 samples at a sampling rate of 250 Hz) of data located between the initial inflection point and a point 40 ms earlier. In this embodiment, the minimum running average may be determined for a number of QRS complexes. The set of running averages may then be analyzed statistically to cull outliers (which could be noisy or ectopic beats). In the remaining set (F) of representative average flatnesses, the average flatness may then be taken as the desired flatness level. In some cases, it may be desirable to err on the side of choosing a relatively higher flatness level (e.g. take the maximum instead of the average of set F) while in others the contrary may be true; generally, the impact of a too lenient or too strict a flatness criteria depends on expected QRS morphology and the details of the QRS onset/offset stopping criteria, as will be further described below.

Block 622 calculates two different curvature function noise levels. The preferred curvature function is equal to the local second derivative squared. (Alternatively, the second derivative may be multiplied by its most significant binary bit, which is akin to squaring the second derivative but is computationally more efficient since it can be effected with a simple shift left of the binary value of the second derivative.) For a discrete valued signal S(i), where i is an integer, the preferred raw second derivative at sample i is: S(i+1)−2*S(i)+S(i−1). The preferred actual second derivative is based on the above formula but suitably modified to take into account signal averaging.

The curvature function values are used both to detect a QRS complex (“QRS Noise Level”) and, once a QRS has been found, to aid in the search for its onset/offset points (“Onset/Offset Noise Level”). The QRS Noise Level is the threshold above which a sample is reasonably likely to be within a QRS complex, as opposed to a T-wave. T-waves frequently have the highest values of the curvature function outside of the QRS. Thus, the appropriate QRS Noise Level to eliminate T waves from consideration as QRS' is the maximum value of the curvature function associated with a T wave. (In some cases, P waves may have greater curvature than T waves, in which case P wave curvature should be examined instead of T wave curvature.)

The QRS Noise Level may be found by examining a 5 second segment of signal S, which is very likely to have at least 3 QRS', locating the maximum value of the curvature function, which is likely to be within a QRS, and then finding the maximum value of the curvature function in the likely region of the T wave, which is between 100 ms and 400 ms after the curvature function maximum point. The values of another T wave curvature function maximum may be found by excluding the previously located QRS from the signal, finding the remaining maximum of the curvature function (within another QRS), and then finding the T wave curvature maximum associated with that QRS. The QRS Noise Level may then be taken as the maximum over all of the located T wave curvature function maxima.

An example of a curvature function noise level for the detection of QRS onset/offset (Onset/Offset Noise Level) is shown in FIG. 7. The curvature function values of signal S drop below this threshold outside of the major inflection points of the QRS, so this threshold will be used (as further described with reference to FIG. 5) to find the region near the QRS onset/offset point. If the signal S dynamic range is scaled from 0 to 1000, an empirically derived noise threshold of 16 yielded good results across a variety of ecg waveforms (at 250 Hz). The Onset/Offset Noise Level may also be derived adaptively in a variety of ways by a relatively computationally expensive process that is performed periodically. For a number of QRS', the PQ and/or ST segments may be located by finding a string of 3 or 4 samples that are flat and adjacent to an initial or final QRS inflection point. The maximum curvature function in the two or three samples adjacent to the flat region (and toward the Q wave) may then be taken as the Onset/Offset Noise Level.

Another alternative to compute the Onset/Offset Noise Level is to find the maximum curvature function value for a number of QRS complexes. Empirically, the appropriate noise level is approximately two orders of magnitude less than the maximum curvature value.

Block 624 initiates the process of locating possible QRS complexes by setting a starting search point at somewhere between 100 ms and 200 ms after the beginning of the current segment. The 100 ms-200 ms offset helps to ensure that the search does not locate the interior of a QRS that starts the segment but whose onset is outside (before) the segment. In block 626, a QRS threshold test, which is a stopping test, is applied by comparing the curvature function value of the current sample (S(i)) with the QRS Noise Level. If S(i) is less than the QRS Noise Level, the routine moves to block 632, where it hops ahead Na samples, which is preferably 4 samples (at 250 Hz).

If a QRS has not been detected within an expected number of samples, the QRS Noise Level may be temporarily reduced until the next QRS is located. The extent of the reduction preferably depends on the difference between the number of samples that have passed without a QRS being detected and the expect number of samples, which is a function of heart rate. For example, at a heart rate of 60 beats/minute, a QRS is expected roughly every 250 samples (at 250 Hz sampling). The QRS Noise Level (Nqrs) may reduced from its baseline value to a minimum of half that value according a function such as Nqrs/min(max(j−250),1),2), where j is the number of samples that have passed since the last QRS was detected.

Returning to block 626, if the curvature function value at the current sample exceeds Nqrs, then control passes to block 627 to verify that the possible QRS region located in block 626 is within a QRS. Block 626 performs a verification test by calculating the curvature function value at 10 samples after the current sample. If the average curvature function value of the 11 samples (the current sample along with its 10 neighbors to the right) exceeds Nqrs, control passes to block 628 for additional tests to determine whether the current region is within QRS. Otherwise, control again passes to block 632.

In block 628, yet another verification test (to help eliminate high frequency, low amplitude noise) is applied by examining the long distance derivative in a region starting with the current sample and ending n samples later, i.e, long distance derivative=S(i+n)−S(i). The threshold for determining whether a long distance derivative qualifies for QRS status is best determined empirically; given the scaling and flatness criteria adopted in the code set forth in FIGS. 9a-9l, a threshold value of 10*Flat*n yielded good-results. The long distance derivative is preferably examined to the left (e.g. n equals −5) of the current sample.

If the long distance derivative test fails, control again passes to block 632. Otherwise, in block 629, the QRS onset/offset points associated with the current point are obtained in the manner described in FIG. 5. Control passes to block 630, which checks whether the QRS onset/offset pair falls within the range of a physiologically reasonable QRS complex duration. If so, the current onset/offset pair is stored and Nb samples are skipped, where Nb is the minimum number of samples within which a QRS is very unlikely to occur. A good value for Nb is 50, which corresponds to a hop of 200 ms. If the QRS duration is not reasonable, control again passes to block 632.

FIG. 5 is a flow chart that shows the high level steps for computing QRS onset and offset times. In block 650, two parameters are initialized; these parameters help speed the onset and offset search processes, respectively, and will be updated based on information generated during the search processes.

In block 651, the starting point for the onset search is set at a certain number of samples before the current point, which was located by the QRS detection routine (see FIG. 4). For example, assume the true onset point is at 10 and the QRS detection routine found a possible QRS region at 18 (because the 8 samples after sample 18 qualified under the curvature test). In this case, it would be optimal to hop back 8 samples. Of course, the actual onset region is not known so the number of samples to hop back is also not known.

However, after a number of searches, the routine will have generated information regarding an optimal hop back, which is updated in block 654 after each onset point search. In the preferred embodiment, the expected hop back is set so that it is always smaller than the distance between the current point and the onset point. In other words, the initial hop back is set so that it never overshoots the onset point. In an alternative embodiment, overshooting would be allowed (e.g. to sample 6 in the above example), in which case the QRS onset search routine would have to be able to search forward (e.g. so that it finds sample 8 from sample 6) as well as backward (e.g. finding sample 8 starting at sample 12).

The initial hop back is thus determined adaptively, as reflected by the dotted feedback line in FIG. 5 from block 654 to block 651. The initial expected hop back may be set empirically or at some small number. After the first search for an onset point, in block 654 the expected hop back is increased by a fraction (e.g. ⅛) of the total hop backward required in the first search (e.g. 8 in the above example, which is equal to the starting sample 18 minus the true onset point of 10). However, to prevent the expected hop back from becoming too large (e.g. as the result of one particularly unusually large hop back), the expected hop back can never exceed a preset threshold that is preferably empirically determined.

The expected hop backward is gradually increased after each QRS onset point detection in the manner described above, thereby decreasing the net hop required by the onset search routine. Continuing with the above example, if the expected hop back is gradually increased from 1 to 5, then the net hop back will have decreased from 8 (18-10) to 3 (18-5). Eventually, the search for a particular QRS will have required a hop back of 1. When this occurs, the expected hop back is not increased further for the current segment, since any further increases would make it possible to overshoot an onset point.

The same expected hop back point may be used for subsequent segments but may also be updated periodically and/or due to changes in heart rate or other patient state changes (e.g. posture). The expected hop back could also be made a function of beat type (e.g. normal sinus rhythm beat or ectopic beat).

A similar feedback procedure, implemented by blocks 656, 658 and 660, determines the optimal starting point for the QRS offset search. In this case, the required hop forward from offset to onset point (i.e. QRS duration) for each search is used to adjust the expected hop forward, which eventually will become close to (but slightly less than) the QRS duration. The expected hop forward may be performed by simple averaging. The initial hop forward of 80 ms (block 650) is set so that it is less than, but reasonably close to, expected QRS duration.

Again, the expected hop forward may depend on beat type; in the preferred embodiment, the likelihood that a beat is ectopic is determined by the QRS onset search routine and the expected hop forward is adjusted accordingly upon such a determination. Also, the expected hop forward, as with the expected hop backward, may be used for subsequent segments but may also be updated periodically and/or due to changes in heart rate or other patient state changes (e.g. posture).

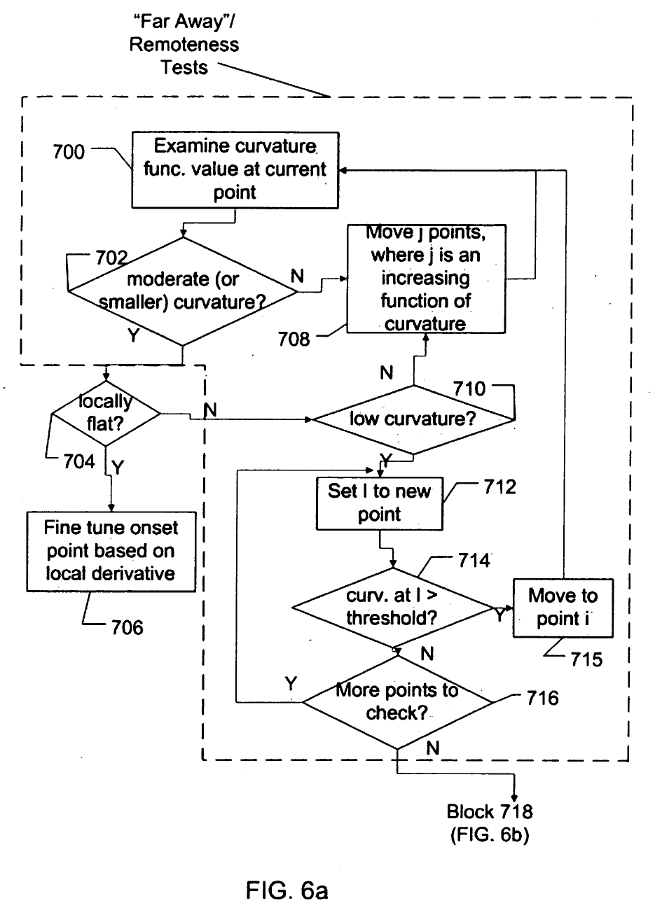

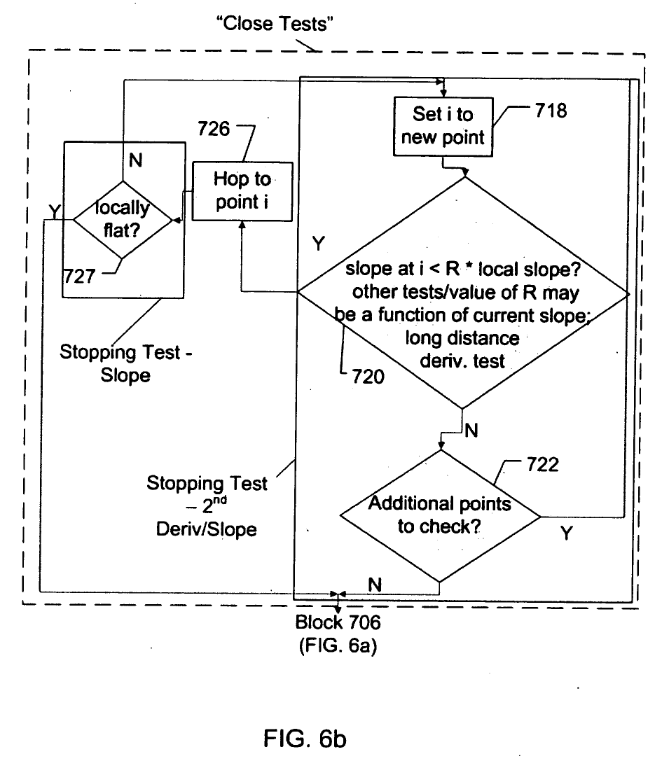

FIGS. 6a and 6b together are a block diagram of the details of the QRS onset/offset detection routine, which is fed a starting search point from the high level routine shown in FIG. 5. In outline, the routine finds onset/offset points by moving toward regions of lower curvature, through the blocks enclosed by a box labeled “Far Away”/Remoteness Tests. Once the routine reaches an region where the curvature locally and further ahead (in the direction of the search) is low, then it applies a series of tests (labeled “Close Tests”), based on local slope (first derivative), that are appropriate for determining the region of a coarse onset/offset point.

The “Close Tests” find a coarse onset point by locating a flat waveform region or by locating a waveform region at which the slope can not be lowered substantially by additional moves in the search direction. In summary, either local flatness or “deceleration” (slowing in the change of slope) serve as the search stop criteria.

The various searches described above are performed by applying tests to selected samples, as opposed to searching sample by sample through the waveform, thereby allowing the routine to hop ahead during a search. The size of a hop may be determined based upon a local function value, further reducing computational load. For example, if the curvature at a certain region is very large, it may make sense to make a large hop, based on the assumption that the QRS marker point being searched for is far away.

An example of the detection of an onset point may help to describe the outlines of the onset/offset region procedure. FIG. 7 shows graphs 800, 802 and 804 of the initial portion of a QRS complex, its corresponding first derivative, and its corresponding curvature function value, respectively. The solid line indicates the current point/region, labeled A, located by the QRS detection routine. The onset/offset detection routine examines the curvature function value at point A and iteratively/sequentially moves ahead (towards the onset point, in the direction of the arrow near A in graph 804) until the curvature is smaller than the Onset/Offset Noise Level, which is indicated by a dotted line. (In the description of onset/offset point region, “ahead” will always refer to the search direction, which is backward in time for onset points and forwards in time for offset points.)

This threshold is reached at the region indicated by the dashed line. After this point has been reached, the coarse onset point is detected by finding a flat region (in the absolute sense), or if none exists (which may especially be the case in offset detection, depending on waveform morphology), the point where the slope (graph 802) changes less than a threshold (i.e. the signal decelerates). In the case of the waveform in graph 802, a flat region was located, indicated by a dash-dot line.

Turning to the details, block 700 in FIG. 6a examines the curvature function value at the current point (e.g. point A in FIG. 7). Block 702 moves to block 704 if the local curvature function value is small enough to indicate that the current point could be near the QRS onset point. More particularly, block 702 moves to block 704 when the curvature function value is less than a multiple of the Onset/Offset Noise Level (see block 622 of FIG. 4). A multiple of 10 has produced good results. In block 704, the local slope over a region of 16 ms is examined. If the slope over a local 16 ms region is less than a threshold (a function of Flat), then the search is over, and block 704 moves to block 706. A threshold of 20 (10 times Flat, when Flat=2) has produced good results.

Preferably, adjacent 16 ms regions in the region of the current point are examined for flatness. For example, if the current point is 10 and the search is for an onset point (somewhere before 10), the regions from 9-12, 8-11, 7-10, and 6-9 are all examined for flatness. More generally, the flatness search is performed within a 7 sample region (samples 6-12 in the above example). Before examining the entire 7 sample region, a first derivative threshold test may be applied to the current point and the remainder of the region may be searched only if the first derivative is below the threshold.

If none of the regions examined above is below the above mentioned flatness threshold, then block 704 moves to block 710. If the curvature is not low, the onset/offset point is likely a fair distance ahead, so an appropriately sized hop is made in block 708. The number of samples to hop ahead may be curvature dependent (i.e. greater hops when the curvature is larger). Alternatively, a fixed hop may be taken. Block 708 is also passed to from block 702 if the latter determined that the local curvature was greater than moderate. In this case, since the routine has moved to block 708 from block 702, the curvature function value of the current point is known to exceed some multiple of the Onset/Offset Noise Level, as established in block 702. Given this information, a fixed, relatively large hop size (e.g. 16 ms) is taken. Block 708 passes control back to block 700.

Returning to block 710, if the curvature is low, it is possible that the current point is in a curvature “valley” and that there are regions of larger curvature ahead. (An example of such a valley is sample 56 in waveform 804 in FIG. 7).

To climb out of any such valley, the curvature function value at selected samples ahead is examined. This examination is performed by the combination of blocks 712, 714 and 716. First, the curvature of a “far away” sample is checked. (E.g. if the current sample is 56 in waveform 804, the sample at 51 may first be examined.) Block 712 sets the region of this distant sample and block 714 compares the curvature of this sample to the Onset/Offset Noise Level. If the curvature exceeds the Onset/Offset Noise Level, then the distant sample becomes the new current sample by hopping ahead in block 715, to which control is passed from block 714.

If the curvature function value of this distant point does not exceed the Onset/Offset Noise Level, a closer point (or points) is compared to this threshold by blocks 716, 712, and 714. Returning to the above example, sample 54 is checked since sample 51 was below threshold. Since sample 54 is above threshold it becomes the new current point.

Note that in this example, it actually would have been better to hop to sample 51 rather than sample 54. However, an algorithm that involved hopping to a “far away” (e.g. sample 51) below threshold sample could resulting an overshoot of the onset point. For example, if the curvature in between samples 50 and 56 was all below threshold, and the slope between samples 52 and 55 was flat, if sample 51 was hopped to from sample 56, the true onset point (somewhere between samples 52 and 55) would have been skipped over. In an alternative embodiment, such skipping over would be permissible and coupled with a search for the onset point in the opposite direction (e.g. from sample 51, samples 52-55 may be examined as potential onset points.)

Returning to the flow chart, if none of the samples examined (e.g. samples 51 and 54) exceeded the Onset/Offset Noise Level, then the routine has likely reached a relatively low frequency region of the QRS beyond any significant QRS maxima/minima points (which are associated with high curvature/frequency). For example, if the current point is sample 52 (dashed line), which is just beneath the Onset/Offset Noise Level, there are no regions of large curvature ahead because the routine has passed the high frequency bend in the neighborhood of sample 55. At this point, starting at block 718 and indicated by the dotted box labeled “Close Tests”, the routine applies two different stopping tests, labeled as “Stopping Test—Slope” and “Stopping Test—2^{nd} Deriv./Slope”, to locate the coarse onset/offset point.

The routine looks for flat (small first derivative) regions in block 718 labeled as “Stopping Test—Slope”. If it can not find any flat regions, in blocks 718-720 labeled as “Stopping Test—2^{nd} Deriv./Slope”, it chooses the onset/offset point based on “deceleration” of the signal, i.e. it finds a sample where the ratio of first derivatives before and after the sample is small.

It finds such a sample by checking if the ratio of the first derivative of the current sample to the first derivative at a various samples ahead of the current sample is large. For example, if the current sample is S in the waveform 802 (FIG. 7), its first derivative is 40. The first derivative of a sample (e.g. sample 52) a few samples ahead of S is approximately 20, yielding a ratio of first derivatives of 2, which is large enough to cause the routine to hop ahead. Had the ratio been 1, the routine would not have hopped ahead. The routine examines a number of samples ahead (e.g. sample 52, sample 50 etc.); if it does not hop to any of them, the signal has decelerated sufficiently in the region of the current sample, which is chosen as a (coarse) onset point.

Turning to FIG. 6b, in block 718, a sample i is selected and its slope examined in block 720. In the preferred embodiment, i is initially set to 2 so that the slope 2 samples ahead is examined. The relatively small value of 2 prevents the routine from skipping over very short flat regions (e.g. on the order of 12 ms) that can result, for example, from the P wave downstroke occurring very shortly before the Q wave. If a particular lead does not strongly register P waves, a larger initial look ahead (e.g. 4 samples so that I=current sample+4) may be appropriate.

In block 720, the slope at sample i is compared to the current slope. If the slope at i is less than R times the current slope, the routine moves to point i in block 726. In the preferred embodiment, if i is relatively close to the current sample, e.g. either 2 or 4 samples away from the current sample, then R is set to 2. Thus, a short hop (i.e. 2 or 4 samples) will be made from the current sample if the slope can be halved by such a hop.

In an alternate embodiment, the value of i may be chosen according to the current slope. For example, if the current slope is relatively large, it may be preferable to first examine the slope at a sample “far away” based on the assumption that a flat region is more likely to occur further away. Conversely, if the current slope is relatively small, a smaller hop forward may be appropriate based on the assumption that a flat region is closer. However, whether or not it is optimal to set the look ahead value (i) according to slope will depend on expected waveform morphology.

Relatedly, the optimal order of samples to examine depends on expected waveform morphology. In the preferred embodiment, close samples are examined before further away samples to avoid skipping past the onset point. Again, as mentioned above, in an alternative this order may be reversed depending on the slope.

If the slope at various points i (e.g. samples 2 and 4 after the current sample) is not less than ½ the local slope, block 718 sets i to samples that are still further away (until a sample passes a test or there are no additional samples left). In turn, block 720 applies different tests to these further away samples. In the preferred embodiment, the next sample that is examined is 7 samples away; this sample is hoped to only if the slope of the current point is relatively large (>4*Flat) and the slope at 7 samples away is either (i) less than half the current slope; or (ii) less than or equal to Flat. The local steepness requirement (>4*Flat) helps to ensure that big hops are not taken when the local slope is relatively small. Otherwise, a very gradual decrease in slope, even where the local region is not too steep, could result in a hop.

If the sample at i=current point+7 does not pass the above test, in the case of searching for offset points, similar tests are successively applied at 10 samples away and then at 16 (if 10 fails). These further away tests are not applied to offset points because such large hops will not generally be appropriate in the PQ segment. For offset points, again, the local steepness criterion is applied (4*Flat) and the slope at these further away samples must be either (i) less than ¼ the current slope; or (ii) less than or equal to Flat.

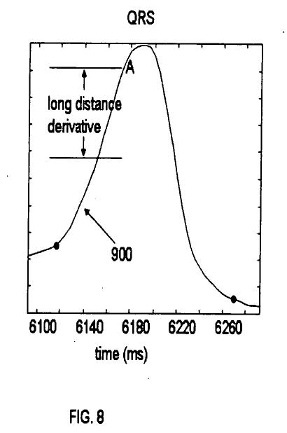

Finally, if none of the above tests is passed, it is very likely that there is no significant deceleration of the signal after the current point. However, in the case of ectopic beats, which can have a very slow upstroke, it still may be appropriate to take a large hop. The situation is illustrated in FIG. 8, which shows a QRS 900 corresponding to an ectopic beat. In this case, assuming the current point is A, the curvature/deceleration after A (i.e. before approximately 6180 ms) is relatively small because the upstroke is gradual. Thus, at point A, all of the aforementioned curvature and deceleration tests may fail but it is apparent that the onset of the QRS has not been reached.

To handle this problem, the long distance derivative between A and a point 10 samples (40 ms) after A is examined. If this long distance derivative is greater than a threshold, 40*Flat(=an average slope of 4*Flat), then a hop of 10 is made. Otherwise, a similar test is applied for a sample 20 samples after A and a hop of 20 is made if the test is passed. The long distance derivative is preferably examined only in the search for QRS onset points. Also, an ectopic flag is set if either of the above long distance derivative tests pass; this flag may be used to set an appropriate starting point to look for the QRS offset (see block 656 of FIG. 5). Relatedly, in an alternate embodiment, the long distance derivative tests are applied only if the start search time for a beat indicates it is likely to be an ectopic beat.

Whenever any of the above described deceleration/long distance derivative tests is passed, block 720 passes control to block 726, which hops to point i. From block 726, control is passed to block 727, which is identical to block 704. If a flat region is found, a coarse onset point has been located and control passes to block 706. Otherwise, control passes back to block 718.

If all of the above series of tests have failed, then the 2^{nd} Deriv./Slope Stopping Test has been passed, and the current point is taken as the coarse onset/offset point. Block 722 passes control to block 706.

Block 706 receives all coarse onset/offset points and attempts to find a better onset/offset point in the region of the coarse point by finding the sample in this region with the smallest slope, subject to a distance based weighting that favors the current point. For example, if the slopes of the current point and three succeeding points are [5 3 2 2], a weighting vector [1 2 2 3] is applied to yield distance weighted slopes equal to [5 6 4 6]. In this case, the second sample after the current sample, which has a slope of 2 and a weighted slope of 4, is selected. Finally, in the case of offset points, if the QRS duration as measured from the current point is much longer than expected, instead of searching the three samples after the current point, the fine tuning is performed on the three samples before the current point.

### ALTERNATIVE EMBODIMENTS

The preferred embodiment has been described with respect to applying tests to a waveform that is representative of an electrocardiogram. However, the teachings of the present invention may be applied to a transformed electrocardiogram waveform (e.g. as described in Arzeno et al., Analysis of First-Derivative Based QRS Detection Algorithms, IEEE Transactions on Biomedical Engineering, Vol. 55, No. 2, 2008) that is characterized by high frequency regions associated with internal QRS components and a flat and/or diminishing slope at QRS onset/offset points. Still more broadly, the teachings of the present invention may be applied to QRS marker points generally, not just onset/offset points.

If a QRS starting search point occurs early as determined by comparing the times between other QRS starting points (similar to an R-R interval analysis as described, for example, in U.S. patent application 20070093720, System for detection of different types of cardiac events, to Fischell et al., then it may be desirable to search for QRS offset points first, and then search for the onset point. (The offset points in ectopic beats may be better defined than the onset points in the case where an ectopic beat occurs just after a T wave.)

The proper amount of hopping (e.g. performed by block 708 in FIG. 6) given local curvature is optimally determined empirically. In another embodiment, the signs of the first and second derivatives may be considered along with the value of the preferred curvature function; depending on QRS morphology, this may yield information regarding region in the QRS complex. For example, some QRS morphologies may have a negative derivative at every point after the R wave peak; in this case, an examination of the sign of the first derivative can convey information as to the region of a current point within the QRS complex. (E.g. if the first derivative is negative, the current point is after the R wave, so if it is desired to find the QRS onset point, a large hop may be necessary. Again, the optimal hop size given the curvature function value and first derivative sign may be empirically derived.) The combination of the sign of the first and second derivatives, and the values of the curvature function at the current point and a point (or points) some distance away, may provide relatively better hopping distances at the cost of additional computations to determine the hopping distance.

Although the methods shown in FIGS. 5 and 6 have been described with respect to finding QRS onset/offset points, it will be appreciated that these methods may be applied to other waveform marker points (e.g. peak T wave). The routine gettpeak in FIGS. 9j-9l, is fed a starting search point from routine doall, which implements a similar feedback on T peak search distance as was previously described with respect to onset/offset points. The first guess of the starting search point is based on the first R-R interval (implemented as the distance between the first two onset points) and thereafter the starting search point is based on the distances associated with previous searches.

The routine gettpeak searches for points on a rising (or falling) portion of the T wave by applying first derivative criteria and second derivative criteria somewhat similar to the criteria applied with respect to onset/offset points. At each new point in the search, a stopping test is applied based on either flatness or high curvature. The high curvature test is helpful in cases where a T wave peak is very sharp (and thus not flat for a large distance) and is implemented by checking curvature and checking whether the slopes on either side of the point in question have opposite signs, indicative of a peak.

Various other modifications, adaptations, and alternative designs are of course possible in light of the above teachings. Therefore, it should be understood at this time that, within the scope of the appended claims, the invention can be practiced otherwise than as specifically described herein.

What is claimed is:

1. A device for detecting a QRS complex within an electrocardiogram, comprising:
a lead comprising two sensors, the lead adapted to be disposed so that it senses an electrocardiogram,
a processor coupled to the lead, the processor configured to:
(i) obtain a signal from the lead and create therefrom a discrete waveform comprising a plurality of samples;
(ii) search for a QRS complex within the discrete waveform by performing a stopping test at a plurality of regions within the waveform and identifying a possible QRS region when the stopping test is positive, wherein at least two consecutive non-overlapping regions within the plurality of regions are separated by at least one sample and less than an expected minimum QRS duration.

2. The device for detecting a QRS complex according to claim 1 wherein the processor is further configured to apply a verification test to the possible QRS region to determine if it is within a QRS.

3. The device for detecting a QRS complex according to claim 1 wherein the stopping test involves the comparison of a curvature function value with a threshold.

4. The device for detecting a QRS complex according to claim 2 wherein the verification test involves the comparison of a threshold with a function based on the sum of a plurality of curvature function values at a corresponding plurality of regions adjacent to the possible QRS region.

5. A device for detecting a QRS complex within an electrocardiogram, comprising:
a lead comprising two sensors, the lead adapted to be disposed so that it senses an electrocardiogram,
a processor coupled to the lead, the processor configured to:
(i) obtain a signal from the lead and create therefrom a discrete waveform comprising a plurality of samples;
(ii) determining a plurality of regions within the discrete waveform that are within a corresponding plurality of QRS complexes, wherein each of the plurality of regions may be a variable distance from a QRS fiducial point;
(iii) starting from each of the plurality of regions, performing a search for a corresponding QRS marker point that is either a QRS onset point or a QRS offset point.

6. A device for detecting a QRS marker point within an electrocardiogram, comprising:
a lead comprising two sensors, the lead adapted to be disposed so that it senses an electrocardiogram,
a processor coupled to the lead, the processor configured to:
(i) obtain a signal from the lead and create therefrom a discrete waveform comprising a plurality of samples;
(ii) determine a search start region within a QRS complex that is within the discrete waveform;
(iii) starting from the search start region, perform a search for a QRS marker point by computing a first function value at a first QRS region that is a known distance from the search start region, and moving toward the QRS marker point by a distance that is a function of the first function value.

7. The device for detecting a QRS marker point according to claim 6 wherein the first QRS region is the search start region.

8. The device for detecting a QRS marker point according to claim 6 wherein the first QRS region is a specified distance from the search start region.

9. The device for detecting a QRS marker point according to claim 6 wherein the first function value is a function of the curvature of the first QRS region, and wherein the distance is an increasing function of the curvature.

10. A device for detecting a marker point within an electrocardiogram, comprising:
a lead comprising two sensors, the lead adapted to be disposed so that it senses an electrocardiogram,
a processor coupled to the lead, the processor configured to:
(i) obtain a signal from the lead and create therefrom a discrete waveform comprising a plurality of samples;
(ii) determining a start search region that is within the discrete waveform;
(iii) starting from the start search region, moving toward the marker point based on an expected distance between the start search region and the marker point region, thereby moving to an intermediate region;
(iv) starting from the intermediate region, performing a search for the marker point, thereby locating the marker point;
(v) computing a distance between the marker point and the starting search point; and
(vi) based on the distance between the marker point and the starting search point, updating the expected distance between a starting search point region and a marker point region.

11. The device for detecting a QRS marker point according to claim 10 wherein the search start region is a QRS marker point.

12. The device for detecting a QRS marker point according to claim 10 wherein the search start region is an unknown distance from any fiducial point.

13. A device for detecting a QRS marker point within an electrocardiogram, comprising:
a lead comprising two sensors, the lead adapted to be disposed so that it senses an electrocardiogram,
a processor coupled to the lead, the processor configured to:
(i) obtain a signal from the lead and create therefrom a discrete waveform comprising a plurality of samples;
(ii) determining a search start region within a QRS complex that is within the discrete waveform;
(iii) starting from the search start region, performing a search for a QRS onset/offset point by iteratively moving to regions closer to the onset/offset point and stopping the search at a region upon the earlier of:
(i) a first derivative function of the region becoming sufficiently small; (ii) a second derivative function of the region becoming sufficiently small.

14. A device for detecting a QRS marker point within an electrocardiogram, comprising:
a lead comprising two sensors, the lead adapted to be disposed so that it senses an electrocardiogram,
a processor coupled to the lead, the processor configured to:
(i) obtain a signal from the lead and create therefrom a discrete waveform comprising a plurality of samples;
(ii) determine a search start region within a QRS complex that is within the discrete waveform;
(iii) starting from the search start region, sequentially perform a remoteness test at a plurality of regions until a test outcome at a first region indicates that the first region is past all high frequency QRS regions, and thereafter performing a stopping test at a plurality of regions beyond the first region until the marker point has been located.

15. The device for detecting a QRS marker point according to claim 14 wherein the remoteness test pertains to waveform curvature.

16. The device for detecting a QRS marker point according to claim 14 wherein the remoteness test pertains to waveform slope.

17. A device for detecting a QRS marker point within an electrocardiogram, comprising:
a lead comprising two sensors, the lead adapted to be disposed so that it senses an electrocardiogram,
a processor coupled to the lead, the processor configured to:
(i) obtain a signal from the lead and create therefrom a discrete waveform comprising a plurality of samples;
(ii) determine a current region within a QRS complex that is within the discrete waveform;
(iii) starting from the current region, sequentially perform a test at a plurality of regions until the earlier of: (i) a test outcome is positive at a certain region, in which case a new region is moved to; or (ii) there are no further regions to test, in which case the marker point is the current region; and wherein at least two consecutive non-overlapping regions within the plurality of regions are separated by at least one sample.

18. A device for detecting a QRS marker point within an electrocardiogram, comprising:
a lead comprising two sensors, the lead adapted to be disposed so that it senses an electrocardiogram,
a processor coupled to the lead, the processor configured to:
(i) obtain a signal from the lead and create therefrom a discrete waveform comprising a plurality of samples;
(ii) determine a current region within a QRS complex that is within the discrete waveform;
(iii) starting from the current region, sequentially moving to a plurality of regions until a current region is reached at which either: a first stopping test outcome is positive or a second stopping test outcome is positive, wherein the first stopping test is applied upon moving to a new region and the second test is performed only if the first test is negative, and wherein the second stopping test identifies a point to move to if the second stopping test is negative; and wherein at least two consecutive regions within the plurality of regions are separated by at least one sample.

19. The device for detecting a QRS marker point according to claim 18 wherein the first test pertains to a flatness criterion of the waveform in the region of the current sample.

20. The device for detecting a QRS marker point according to claim 18 wherein the second test pertains to a plurality of comparisons between the slopes at a plurality of points and the slope at the current region.
